(19) United States
(12) Patent Application Publication
(10) Pub. No.: US 2005/0267015 A1
(43) Pub. Date: Dec. 1, 2005

(54) Method of treatment and composition for inhibiting the production of toxic free radical and reactive oxygen species using metalloproteins found in bacteria

(76) Inventor: Kareem I. Batarseh, Fairfax Station, VA (US)

Correspondence Address: KILYK & BOWERSOX, P.L.L.C., 400 HOLIDAY COURT, SUITE 102, WARRENTON, VA, 20186, US

(21) Appl. No.: 11/121,763

(22) Filed: May 4, 2005

Related U.S. Application Data
(60) Provisional application No. 60/568,105, filed on May 4, 2004.

(51) Int. Cl. A61K 38/16

(52) U.S. Cl. 514/6

(57) ABSTRACT

Compositions and methods for treating, reducing the risk of, or slowing the onset of a neurological disorder or pathological condition in a subject are provided. An inhibitor of the formation of free radical or reactive oxygen species is administered in an effective amount to the subject and the inhibitor prevents tissue and cellular damage and/or necrosis by inhibiting the release of free radicals or reactive oxygen species (ROS) that can cause such damage in the subject. The inhibitor may be a bacterial metalloprotein. An exemplary free radical production inhibitor is the metalloprotein rusticyanin, a type I blue-copper metalloprotein found in the aerobic acidophilic iron-oxidizing bacterium Thiobacillus ferrooxidan. A composition containing the inhibitor of the formation of free radical or reactive oxygen species includes a pharmaceutically acceptable carrier.

The present application claims the benefit of U.S. Provisional Patent Application No. 60/568,105, which was filed on May 4, 2004 and which is incorporated in its entirety by reference herein.

### FIELD OF THE INVENTION

The present invention relates to compositions and methods for interfering, preventing and/or reducing the production of free radical levels in mammals. In a particular aspect, the present invention relates to compositions and methods for inhibiting and/or reducing the production of free radicals in mammals by the action of inhibitors found in chemolithotrophic bacteria and to the employment of rusticyanin, a type I blue-copper metalloprotein found in T. ferrooxidans, which oxidizes ferrous iron to ferric iron thereby precluding the formation of ROS through the competition for ferrous iron with physiological processes in hosts afflicted with a variety of disorders.

### BACKGROUND OF THE INVENTION

The opportunity gained from the utilization of atmospheric oxygen by the evolving biota as the terminal oxidant in respiration offered energetic advantages over fermentation and other pathways that depend on other oxidants. However, the presence of intracellular oxygen has resulted and allowed inadvertent redox reactions by free radicals to detrimentally affect critical biomolecules, and thereby cause a plethora of human diseases and conditions. A free radical is a molecule that has one or more unpaired electrons represented by a dot (A.) next to the chemical structure. Reactive oxygen species (ROS) are generated by a myriad of normal biological process, including photolysis, thermal homolysis, mitochondrial electron transfer, and radical-forming redox reactions. Because of their high instability and reactivity, overproduction or inappropriate production of free radical species can be detrimental. Such diseases that are attributed to ROS include atherosclerosis, ischemic trauma of the vital organs, in particular, of the central nervous system including the brain, e.g., stroke and its sequelae, reperfusion injury following stroke or acute myocardial infarction (McCord, N. Engl., J. Med., 312, 159 (1985)), and a variety of neurodegenerative disorders (Evans, Br. Med. Bull., 49, 577 (1993); and Day, M., New Scientist, 19 (1999); Ames, B., et al., Proc. Natl. Acad. Sci. USA, 90, 7915 (1993); Ames, B., et al., Proc. Natl. Acad. Sci. USA, 91, 10771 (1994); and Ames, B., et al., Proc. Natl. Acad. Sci. USA, 92, 5258 (1995)), including cancer, aging, Alzheimer's (AD), Parkinson's (PD), and Huntington's (HD) diseases, multiple sclerosis (MS), and progressive supranuclear palsy (PSP).

In addition, considerable biochemical, physiological and pharmacological evidence supports the occurrence and importance of ROS in cardiac ischemia/reperfusion injury (Meerson, F. Z., et al., Basic Res. Cardiol, 77, 465 (1982); and Downey, J. M., Ann. Rev. Physiol., 52, 487 (1990)). It has been proposed that reoxygenation of ischaemic myocardium leads to generation of O.₂ and H₂O₂ within the tissue which can, in the presence of transition metal ions, become converted into highly-reactive hydroxyl radicals (.OH), leading to changes in cell membrane integrity and tissue injury. Exposure of myocytes or whole heart to oxidant-generating systems produced severe injury, including inactivation of the ATP-dependent Ca⁺² sequestering system of cardiac sarcoplasmic-reticulum.

Given the fact that there is a deluge of diseases and traumas associated with the production of ROS, there is an immediate need for the development of pharmaceutical agents or methods which prevent such diseases, and the protection of vital organs of patients in post-traumatic recovery from organ ischemic reperfusion injury. Hence, effective compositions and methods to reduce and minimize the production and release of ROS in patients suffering from a variety of disparate disorders would be a great boon to medicine and serve to reduce and eliminate a substantial amount of human suffering.

Although various methods and techniques have been developed to offset the production of free radicals, such methods suffer from many disadvantages. The majority of treatments for excess free radical production available in the literature involve the use of scavengers or enzymatic inhibitors to reduce radical production, or the use of protein-based antioxidants, such as superoxide dismutase, to neutralize and reduce the potential cytotoxic effects of free radicals (e.g., Crapper-McLachlan, D. R., et al., Lancet, 337, 1304 (1991); Friden, P. M., et al., Science, 259, 373 (1993); and United States Published Application No. 20030040511). These approaches have proved unsatisfactory, because by the time a patient shows symptoms and can be treated, substantial amounts of free radicals have already been generated. Hence, there is the need to develop a method that can overcome these deficiencies.

### SUMMARY OF THE INVENTION

In accordance with the present invention, there are provided compositions and methods for preventing tissue and cellular damage and/or necrosis that relates to the inhibition and/or reduction of the release of free radicals or reactive oxygen species (ROS) in mammalian subjects in need thereof. Contrary to the scavenging approach described in the prior art (i.e., overproduced free radicals are bound to suitable free radical scavengers), the present invention relies on interfering, terminating and/or reducing the effect of the function of the species responsible for free radical production. This is accomplished by the introduction of inhibitors, competing agents, procured from chemolithotrophic (involves electron transport pathways which oxidize an inorganic compound) bacteria which are capable of oxidizing metal ions as their energy source for growth so that such metal ions are not available for the physiological production of ROS in hosts where such therapy is needed. In essence, this invention relies on depleting the metal ion pool so metals are not available for ROS production. The methods of the present invention are useful in preventing and treating a variety of neurological diseases such as those encountered with Alzheimer's disease, Parkinson's disease, progressive supranuclear palsy, cystic fibrosis, multiple sclerosis, and the like, or pathological situations such as ischemic trauma, strokes and its sequelae, cancer, aging, transplant rejection, AIDS dementia, vascular and aortic aneurysms, myocardial infarction, and the like, which are associated with ROS production and release. An exemplary free radical production inhibitor contemplated for use in the practice of the present invention is the metalloprotein rusticyanin, a type I blue-copper metalloprotein found in the aerobic acidophilic iron-oxidizing bacterium Thiobacillus ferrooxidans, T. ferrooxidans. This bacterium converts ferrous iron to ferric iron through the agency of rusticyanin, which is found in the periplasm of the membrane as the energy-generating mechanism for its growth thereby competing for ferrous iron, precluding the presence of the catalytic activities of ferrous iron, which is essential for the physiological production of ROS.

Accordingly, the present invention relates to a method of treating, reducing the risk of, or slowing the onset of a neurological disorder or pathological condition, by administering to a subject in need thereof an inhibitor of formation of free radical or reactive oxygen species in the subject. In particular, the inhibitor reduces a risk of cellular damage mediated by free radical or reactive oxygen species such as .O⁻₂, H₂O₂ or .OH, and particularly, hydroxyl radicals.

The inhibitor may be a bacterial metalloprotein, such as a metalloprotein found in chemolithotrophic bacteria, particularly a metalloprotein found in Thiobacillus ferrooxidans. In particular, a metalloprotein may be selected that promotes conversion of ferrous iron to ferric iron in the subject and has an inhibitory effect on the formation of .O⁻₂, H₂O₂ or .OH. The metalloprotein may a type I blue copper protein and may be azurin, plastocyanin, amicyanin, stellasyanin, umecyanin, or rusticyanin, or mixtures thereof. As a non-limiting example, the metalloprotein may be rusticyanin.

The method of the present invention may also include administering a scavenger of reactive oxygen species, in addition to the inhibitor of the formation of free radicals or reactive oxygen species. The scavenger of reactive oxygen species may be catalase, superoxide dismutase (SOD), ascorbate peroxidase, or glutathione peroxidase.

The present invention further relates to an inhibitor of formation of free radical or reactive oxygen species and a pharmaceutically acceptable carrier. As discussed above, the inhibitor may be a bacterial metalloprotein, such as a metalloprotein found in chemolithotrophic bacteria, particularly a metalloprotein found in Thiobacillus ferrooxidans. In particular, a metalloprotein may be selected that promotes conversion of ferrous iron to ferric iron in the subject and has an inhibitory effect on the formation of reactive oxygen species such as .O₂, H₂O₂ or .OH. The metalloprotein may a type I blue copper protein and may be azurin, plastocyanin, amicyanin, stellasyanin, umecyanin, or rusticyanin, or mixtures thereof. As a non-limiting example, the metalloprotein may be rusticyanin.

The pharmaceutically acceptable carrier of the composition may be a biological buffer, such as a biological buffer that lacks available aqua coordination sites. As a particular non-limiting example, the pharmaceutically acceptable carrier may be diethylenetriamine pentaacetic acid.

The composition may further include DNA-intercalating agents and agents that block activity of topoisomerase I and topoisomerase II. The composition may further include cytokines, antimicrobial agents, antiviral agents, hormones, sedatives, hypnotics, tranquilizers, topically active drugs, vasodilating substances, biological substances that affect tissue formation and tissue stabilization, sunscreens, cleansing agents, or antiperspirants. The composition may further include at least one additional electron source such as a thiol, ascorbate, paraquat, anthraquinone, quinone, semiquinone, redox-activating drugs, antibiotic agent, antitumor agent, bleomycin, amsacrine, mitomycin C, adriamycin, actinomycin D, daunomycine, neocarsinostatin, steptonigrin, elliptinium acetate, or mixtures thereof. The composition may further include scavengers of reactive oxygen species such as catalase, superoxide dismutase (SOD), ascorbate peroxidase, or glutathione peroxidase. The composition may further include at least one metal chelator.

Additional features and advantages of the present invention will be set forth in part in the description that follows, and in part will be apparent from the description, or may be learned by practice of the present invention. The objectives and other advantages of the present invention will be realized and attained by means of the elements and combinations particularly pointed out in the description and appended claims.

It is to be understood that both the foregoing general description and the following detailed description are exemplary only and are not restrictive of the present invention, as claimed. All patents, patent applications, and publications mentioned above and throughout the present application are incorporated in their entirety by reference herein.

The accompanying drawing, which is incorporated in and constitutes a part of this application, serves, together with the description, to explain some of the principles of the present invention.

### BRIEF DESCRIPTION OF THE DRAWINGS

FIG. 1 is a schematic diagram showing the overall reaction mechanism for hydroxyl radical production in biological systems.

### DETAILED DESCRIPTION OF THE INVENTION

The present invention is broadly directed to compositions and methods developed for the interference, prevention, and/or the reduction of excess production of free radicals in subjects in need thereof. In contrast to the treatment approaches previously described, this invention relies on the introduction of inhibitors for the competition of ferrous iron oxidation to ferric iron between biological redox reactions and metalloproteins found, for example, in the iron-oxidizing bacteria such as T. ferrooxidans whereby produced free radicals are inhibited or significantly reduced which renders lesser levels of free radicals production. This method can be employed for the treatment of subjects afflicted with infectious, inflammatory, and/or neurodegenerative diseases, and/or where therapeutic excessive production of ROS is needed.

Aerobic organisms, which derive their energy from the reduction of oxygen, routinely generate amounts of .O₂, H₂O₂, and .OH that inevitably form during the metabolism of oxygen, especially in the reduction of oxygen by the electron transfer system of mitochondria. These three species, referred to as reactive oxygen species (ROS), are used to kill pathogenic microorganisms, and a major contributor to neutrophil-mediated cytotoxicity (Hurst, J. K. & Barerette, W. C., Jr. CRC Crit. Rev. Biochem. Mol. Biol., 24,271(1989)).

ROS or free radicals, in particular the hydroxyl radical, is an etiological agent for a wide range of diseases states and/or indications because it can react with all biological macromolecules (lipids, proteins, nucleic acids, and carbohydrates) and can cause significant damage and cytotoxicity (Fridovich, I., Sci., 201, 875 (1978)). Free radical overproduction is associated with a plethora of diseases, indications and conditions such as aging, septic shock, ischemia, diabetes, arthritis (e.g., rheumatoid arthritis), asthma, Alzheimer's disease, Parkinson's disease, multiple sclerosis (MS), cirrhosis, allograft rejection (e.g., transplant rejection), encephalomyelitis, meningitis, pancreatitis, peritonitis, vasculitis, lymphocytic choriomeningitis, glomerulonephritis, ophthalmologic diseases (e.g., uveitis, glaucoma, blepharitis, chalazion, allergic eye disease, corneal ulcer, keratitis), chronic fatigue syndrome, acute renal failure, stroke, cancers (e.g., breast, melanoma, carcinoma, and the like), cachexia, myocarditis, autoimmune disorders, eczema, psoriasis, urticaria, cerebral ischemia, systemic lupus erythematosis, AIDS, AIDS dementia, neurodegenerative disorders (e.g., chronic neurodegenerative disease), cystic fibrosis, amyotrophic lateral sclerosis, Huntington's disease, hyperphagia, solid tumors (e.g., neuroblastoma), hematologic cancers, liver disease (e.g., chronic hepatitis C), drug-induced lung injury (e.g., paraquat), transplant rejection and preservation vascular aneurysm (e.g., aortic aneurysm), myocardial infarction, cerebral vasospasms, and the like.

The mechanism of formation of ROS at physiological pH includes the activation of a membrane-bound enzyme (NAD(P)H)-dependent oxidase system. This complex reduces oxygen to superoxide anion (.O⁻₂), which rapidly dismutes through the agency of metalloenzymes superoxide dismutases (SOD) to form hydrogen peroxide (H₂O₂) where it can then be converted catalytically to hydroxyl radicals (.OH). Superoxides and hydrogen peroxide are relatively mild oxidants that belie the severity of their direct involvement on damaging organic substrates; hence, their inherent cytotoxicity is attributed to their intracellular generation of .OH. The hydroxyl radical is an extremely powerful oxidant that reacts at nearly diffusion-controlled rates with organic biomolecules.

The overall mechanism can be represented by the following reaction scheme: 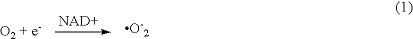
The reduction of molecular oxygen with oxidized nicotinamide adenine dinucleotide (NAD⁺), Reaction 1, occurs very rapidly to form the free radical superoxide oxygen species. The coenzyme (a prosthetic group) NAD⁺ is the most common hydrogen acceptor and oxidizing agent in biological processes: NAD⁺+2e⁻+2H⁺→NADH+H⁺. The molecule contains two major parts, namely, a) an adenosine diphosphate portion, linked through a ribose to b) nicotinamide ring. It is the nicotinamide ring that can be reduced readily and serves as an oxidizing agent.

Superoxide can act as an oxidant (by accepting electrons) or as a reductant (by donating electrons). It potentiates .OH production by two paths, namely, by reducing Fe+3 and recycling the available free iron to Fe+2, and releasing free iron from iron stores such as ferritin. Under biological conditions, the main reaction of superoxide is to react with itself to produce hydrogen peroxide and oxygen, a reaction known as “dismutation”. Superoxide dismutation can be spontaneous or can be catalyzed by a metalloenzyme superoxide dismutase (SOD); thus, 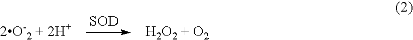
a metal such as Cu, Mn, and Fe mediates the catalytic activities of SOD. Subsequently, the hydrogen peroxide formed can then react with ferrous iron to produce hydroxyl radicals with the aid of SOD according to:
.O⁻₂+Fe^{+3 SOD} Fe⁺²+O₂ (3)

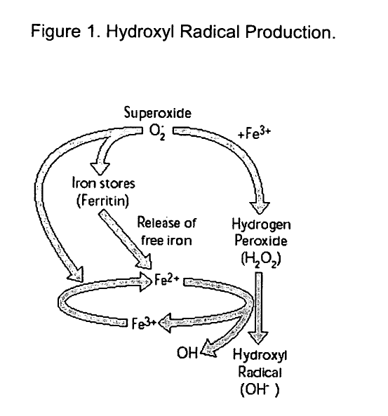

In this process, superoxide acts as a reducing agent since it donates one electron to reduce the ferric iron that acts as the catalyst to convert hydrogen peroxide (H₂O₂) into the hydroxyl radical (.OH). The reduced metal (ferrous iron or Fe+2) then catalyzes the breaking of the oxygen-oxygen bond of hydrogen peroxide to produce a hydroxyl radical (.OH) and a hydroxide ion (—OH); thus,
H₂O₂+Fe⁺²→Fe⁺³+.OH+⁻OH (4)
This reaction is known as the Fenton reaction (Fenton, H. J. H., J. Chem. Soc., 65, 899 (1894)). Hence, Reaction 4 generates ferric lixiviant for Reaction 3 thereby establishing a cyclic process. Reactions 3 and 4 are commonly referred to as the Haber-Weiss reaction (Haber, F. & Weiss J., Proc. R. Soc. London Ser. A, 147, 332 (1934)). The overall reaction mechanism is shown schematically in FIG. 1.

Accordingly, it can be concluded from the above that to maintain an ongoing iron-mediated Fenton reaction, a reduced iron source must be available thereby generating hydroxyl radicals via Reaction 4.

The underlying mechanism of the present invention is the inhibition and/or the lessening of Reaction 4 through the agency of inhibitors, which compete for the oxidation of metals by employing metalloproteins, such as are found in chemolithotrophic bacteria such as the type I blue-copper rusticyanin found in the iron-oxidizing bacterium T. ferrooxidans. This emanates from the fact that these bacteria also employ the oxidation of metal ions to generate energy for their metabolism. In the case of T. ferrooxidans, this metabolism involves the oxidation of ferrous iron and is carried out at the expense of certain nutrients taken into this bacterium in support of growth. In essence, this bacterium will compete with the ROS generating scheme for ferrous iron, precluding the formation and/or reduction of ROS.

Other bacterial metalloproteins such as azurin, plastocyanin, amicyanin, stellacyanin, umecyanin and the like or mixtures thereof can also be used for the present invention.

As used herein, the term “bacterial metalloprotein” refers to a metalloprotein that is found in bacteria. However, the term is not meant to require that the actual metalloprotein that is used in the composition and methods of the present invention is directly taken from bacteria. For example, bacterial metalloproteins that are taken from commercial sources or that are industrially synthesized may be used.

The amount of metalloprotein added depends on numerous factors, including its function, the time needed to accomplish this function, severity of the disease being treated, and the like. Therefore, it is prudent to know a priori information about the severity of the pathological disorder and host being treated in an effort to administer the proper concentration of metalloprotein. Preferably, the amount of bacterial metalloprotein added is inversely related to ferrous iron and should be at least equal to the reciprocal number of metal atoms present in the designated protein multiplied by the molar concentration of ferrous iron present in solution. For example, rusticyanin that contains 1 Cu⁺² per molecule needs to be added in at least 1:1 molar ratio with respect to ferrous iron present in solution.

Since some metalloproteins exhibit their effectiveness at certain pH values, the compositions further comprise pharmaceutically acceptable buffers such as biological buffers that are nontoxic to cells. It is preferred in the present invention to use biological buffers that exhibit no coordination site that is open or occupied by a readily dissociable ligand such as water, i.e., the buffer lacks available aqua coordination sites such as DTPA, diethylenetriamine pentaacetic acid. This will give a considerable strain in the buffer structure, and will not allow opening another coordination position.

According to one aspect of the present invention, several DNA-intercalating agents can be added as well such as those associated with the ability to poison the enzymes topoisomerase I and topoisomerase II which are responsible for the interconversion of the topological states during DNA transcription and replication, and the regulation of DNA supercoiling. Examples of topoisomerase I poisons include protoberberines alkaloids and their synthetic analogs, coralyne, the benzo[c]phenanthridine alkaloids, nitidine (LaVoie, E. J., et al., The Second Monroe Wall Symposium on Biodiversity, Natural Product Discovery and Biotechnology, Simon Bolivar University, Caracas, Venezuela, January 7-9 (1998); Makhey et al., Bioorg. & Med. Chem., 4, 781 (1996); Makhey et al., Med. Chem. Res., 5, 1(1995); and Janin et al., J. Med. Chem., 18, 708 (1975)), as well as the fungal metabolites, bulgarein (Fujii et al., J. Biol. Chem., 268, 13160 (1993)), camptothecin and its derivatives topotecan and irinotecan, bi- and terbenzimidazoles (Bailly, C., CMC, 7, No. 1, 39 (2000); Kim et al., J. Med. Chem. 1996, 39, 992 (1996); Sun et al., J. Med. Chem. 1995, 38, 3638 (1995); and Chen et al., Cancer Res., 53, 1332 (1993)), indolocarbazole derivatives (Bailly, C., CMC, 7, No. 1, 39 (2000); and Yamashita et al., Biochemistry, 31, 12069 (1992)), and saintopin (Yamashita et al., Biochemistry, 30, 5838 (1991)). Other topoisomerase I poisons are β-lapachone, diospyrin, topostatin, topostin, favonoids, Hoechst 33258 and the like and mixtures thereof. Examples of topoisomerase II poisons include teniposide or epipodophyllotoxin, VP-16 and VM-26, and podophyllotoxin-acridine conjugates-pACR6 and pACR8 (Rothenborg-Jensen et al., Anti-Cancer Drug Design, 16, 305 (2001)), and the like and mixtures thereof.

In particular embodiments, the present formulation may be administered in combination with cytokine such as, but not limited to, interleukin; antimicrobial agents such as, but not limited to, acyclovir, chloramphenicol, chlortetracycline, itraconazole, mafenide, metronidazole. mupirocin, nitrofurazone, miconazole, magainins, cecropins, defensins, oxytetracycline, penicillin, tetracycline, and those compositions described in U.S. Pat. Nos. 6,242,009 and 6,630,172, which are hereby incorporated in their entirety by reference; hormones such as, but not limited to, adrenocorticosteroids, cortisone, cortisol, betamethasone benzoate, betamethasone valerate, desonide, fluocinolone acetonide, halcinonide, and hydrocortisone; sedatives, hypnotics and tranquilizers such as, but not limited to, metandienone, benzocaine, dibucaine, lidocaine, pramoxine hydrochloride and tetracacine, pentobarbital sodium, phenobarbital, secobarbital sodium, carbromal, sodium phenobarbital, reserpine, and thiopropazate hydrochloride; topically active drugs (e.g., local anesthetics or anti-pruritics) such as, but not limited to, isotretinoin, benzoyl peroxide, salicylic acid, and tetracycline; analgesics such as, but not limited to, camphor, and menthol; vasodilating substances such as tolazoline; thrombosis-hampering substances such as heparin; certain biological substances which affect tissue formation and tissue stabilization such as EGF (epidermal growth factor), EGF-URo (EGFurogastron), and somatotropin asellacrine; sunscreens such as hydroquinone, monobenzone; and cleansing agents such as soaps and shampoos, and antiperspirants.

Other electron sources such as thiols, ascorbate, paraquat, anthraquinone, quinone and semiquinone or redox-activating drugs such as antibiotic antitumor bleomycin, amsacrine, mitomycin C, adriamycin, actinomycin D, daunomycine, neocarsinostatin, steptonigrin, elliptinium acetate, and the like or mixtures thereof can all be used.

The composition of the present invention further contemplates administering ROS scavengers in conjunction with the inhibitors described herein. Known scavengers of ROS include the enzyme catalyze, superoxide dismutase (SOD), ascorbate peroxidase, glutathione peroxidase, and those compositions which are taught by Crapper-McLachlan, D. R., et al., Lancet, 337, 1304 (1991), Friden, P. M., et al., Science, 259, 373 (1993), and United States Published Application No. 20030040511, which is hereby incorporated by reference.

In addition, metal chelators can be used as well. Metal chelators that can be used should exhibit no coordination site that is open or occupied by a readily dissociable ligand such as water, i.e., the metal chelator lacks available aqua coordination sites such as Desferal (desferrioxamine B methanesulfonate), phytate, EHPA, ethylenediamine di(o-hydroxyphenylacetic acid), and the like or mixtures thereof.

In the case of in vivo application, certain embodiments of the present compositions are nontoxic to humans and animals, and therefore those of skill in the art recognize that the ROS inhibitors described herein can be safely ingested or administered to human and animal subjects via nasal, buccal, vaginal, rectal, and topical, and can be contacted via subcutaneous, intradermal, intramascular injections or any other effective route to the site of infection. Preferably, the route of administration is designed to obtain direct contact with the pathological disorder being treated. The compositions further comprise pharmaceutically acceptable stabilizers, adjuvants, diluents, bioactive chemicals such as antivirals, antibiotics, vitamins, minerals, and mixtures thereof, and other components that are well known to those skilled in the art. Carriers for topical applications may take the form of liquids, gels, foams, lotions and the like and can comprise organic solvents such as ionic and/or nonionic surfactants, perfumes, dyes and the like, and other ingredients commonly used in the pharmaceutical industry which are approved for such uses. Again, such carriers are preferably inert. The antibiotics, antiviral compounds, the chemotherapeutic agents, and the analgesics can be added singularly to the compositions of the present invention, or in combination with each other.

Also contemplated in the present invention is the inclusion of various antiviral agents. Examples of these agents include 9-(2-Hydroxyethoxymethyl)guanine, ZOVIRAX (GlaxoWelicome), idoxuridine, trifluorothymidine, bromovinyldeoxyuridine, ribavirin, amantadine, rimantadine, nevirapine (NVP), and the like.

As readily recognized to those skilled in the art, the ROS inhibitors contemplated for use herein can be administered in a variety of pharmaceutically acceptable forms, including solids, solutions, emulsions, micelles, syrups, elixirs, liposomes, suppositories, hard gelatin capsules, and the like. They can also be mixed with carriers or excipients suitable for enteral or parenteral applications. In addition, pharmaceutically acceptable agents such as auxiliary, stabilizing, thickening, perfumes, and natural and artificial flavoring and coloring so as to provide elegant and palatable preparations may be added as well. In some embodiments, the pharmaceutical composition may be in the form of sterile injectable suspension. This can be accomplished by known methods available to those skilled in the art using, for example, dispersing, surfactants, suspending or wetting agents.

It will be appreciated that the actual preferred method and order of administration will vary according to the particular pathological disorder and the host being treated. The optimal method and order of administration of the subject composition for a given set of conditions can be ascertained by those skilled in the art using conventional techniques and in view of the information set out herein by the present invention.

Those skilled in the art readily recognize that other metalloproteins found in aerobic or anaerobic bacteria capable of biologically oxidizing iron can be singularly utilized or can be used in combination with other metalloproteins found in other iron oxidizing bacteria, such as, for example, Dechlorosoma suillum found in the work of Lack et al. (Lack, J. G., et al., Microb. Ecol, 43, 424 (2002)).

It will be apparent to those skilled in the art that the inhibitors described herein can be administered via controlled release metering devices. The methods and devices include biodegradable polymers, creams, lotions, liposomes, gels, capsules, pumps, syringes, infusion devices, and the like.

The inhibitors described herein can be used for a plethora of diseases and conditions associated with excess ROS production such as multiple sclerosis, Parkinson's diseases, Alzheimer's disease, septic shock, cancer, ischemia, AIDS (auto immune deficiency syndrome), neurodegenerative disorders (e.g., chronic neurodegenerative disease), cystic fibrosis, Huntington's disease, liver disease (e.g., chronic hepatitis C), transplant rejection and preservation vascular aneurysm, myocardial infarction, cerebral vasospasms, diabetes, infections associated to bacterial, viral, parasitic, and fungal, migraine, obesity and all conditions and diseases where excess ROS is present.

Good candidates for the application of the present invention are neurological diseases such as Alzheimer's disease (AD), Parkinson's disease (PD), Huntington's disease (HD), multiple sclerosis (MS), progressive supranuclear palsy (PSP), Hallervorden-Spatz disease, and the like.

Hence, an aspect of the present invention contemplates a method for treating neurodegenerative diseases in subjects in need thereof. This emanates from the fact that patients with such diseases exhibit high production of ROS. In what follows, the involvement of iron in neurologic diseases, particularly as it relates to oxidative damage will be discussed.

The presence of iron is ubiquitous in the human body since it is an essential component of numerous cellular enzymes and synthesis of many hormones, including the cytochrome oxidases, the citric acid cycle, ribonucleotide reductase, the rate-limiting step for DNA synthesis, NADPH reductase (Wigglesworth, J. M., et al., In Brain Iron: Neurochemical and Behavioral Aspects, Youdim, M., ed., Taylor & Francis, New York, 25 (1988)), dopamine, serotonin, and catecholamines syntheses (Youdim, M., In Brain, Behavior, and Iron in the Infant Diet, Dobing, J., ed., Springer-Verlag, London, 83 (1990)), and myelin formation (Larkin, E., et al., In Brain, Behavior, and Iron in the Infant Diet, Dobing, J., ed., Springer-Verlag, London, 43 (1990)). Although iron is an essential nutrient, it is also a potent toxin since it is involved in the production of ROS as discussed previously. This duality necessitates the evolution of a regulatory system composed of transferrin, transferrin receptors and ferritin that can monitor the timely delivery of iron to cells.

In the human body, including the brain, as well as in most organisms, iron is stored mainly in the core of the iron storage protein ferritin which can store as much as 4500 atoms of iron in the form of biomineral ferrihydrite-a hydrated ferric Fe(III) oxide 5Fe₂O₃.9H₂O. Under normal circumstances, the Fe(III) is rendered unreactive with other molecules within the cell due to the protein cage carrier (Allen, P. D., et al., Biochim, Biophys. Acta, 1500, 186 (2000)). Sequestration of the highly toxic Fe(II) is thought to occur primarily through oxidative processes where Fe(II) is converted to the less toxic Fe(III) in the form of ferrihydrite (Harrison, P. M., et al., Biochim, Biophys. Acta, 1275, 161 (1996)). If there is a disruption of ferritin's function or if the ferritin core becomes overloaded, the mechanism of oxidation of Fe(II) to Fe(III) is lost and iron homeostasis is disrupted which can have dramatic health ramifications. If this occurs, it could lead to the formation of biogenic magnetite, which is more strongly magnetic (ferrimagnetic) than ferrihydrate (a superparamagnetic antiferromagnet at body temperature) and contains alternating lattices of the less toxic Fe(III) and the toxic Fe (II) (Dobson, J., FEBS Letters, 496, 1 (2001)), and oxidative damage and the formation of ROS via Fenton reaction or via triplet state stabilization (Kirschvink, J. L., Phys. Rev., 46, 2178 (1992); Scaiano, J. C., et al., Photochem. Photobiol., 65, 759 (1997); and Chignell, C. F., et al., Photochem. Photobiol., 56, 598 (1998)).

In the brain, iron can be found everywhere, but the highest levels are found in the globus pallidus, caudate nucleus, putamen and the substantia nigra, while lower levels are found in the cortex, and cerebellum (Akoi, S., et al., Radiology, 172, 381 (1989)). Except for the liver, the brain has the highest rate of oxidative metabolism in the body. Accordingly, normal neurological function in the brain requires normal iron homeostasis.

The disruption of iron homeostasis has resulted in a plethora of pathological disorders, and has been suggested and implicated for a number of neurological diseases, including AD, PD, HD, PSP, MS, and microgliosis (Beard, J. L., et al., Nutr. Rev., 51, 157 (1993); Fisher, P., et al., Life Sci., 60, 2273 (1997); Bartzokis, G., et al., Cell Mol. Biol., 46, 821 (2000); and Shoham, S., et al., Cell Mol. Biol., 46, 743 (2000)) which is as a result of oxidative stress and generation of free radicals via Fenton kinetics (Marksbery, W. R., Free Radic. Biol. Med., 23, 134 (1997); Connor, J. R., et al., J. Neurol. Sci., 134, 33 (1995); Smith, M. A., et al., Proc. Natl. Acad. Sci., 94, 9866 (1994); and Sayer, L. M., et al., J. Neurochem., 74, 270 (2000)).

Parkinson's Disease (PD)

Recent studies have focused on the involvement of iron in the development of PD. Data collected on postmortem brain iron of patients who had PD showed that there was a 255% increase in ferric iron in the substantia nigra, and 176% increase in total iron (Sofic, E., et al., J. Neural Transm., 74, 199 (1988)).

Multiple Sclerosis (MS)

In MS, the production and maintenance of myelin, the prototype of demyelinating diseases, is disrupted through the disruption of iron regulatory cells, oligodendrocytes. Studies have shown that the cellular pattern of iron is significantly altered and iron is found within MS plaques, and transferrin, which should be located in the oligodendrocytes is found instead in the astrocytes surrounding MS plaques and in demyelinating regions in central pontine myelinolysis (Craelius, W., et al., Arch. Pathol. Lab. Med., 106, 379 (1982); Gocht, A., et al., Acta Neuropathol., 80, 46 (1990); Esiri, M., et al., Neuropathol. Appl. Neurobiol., 2, 233 (1976); and Drayer, B., et al., Am. J. Neuroradiol., 8, 413 (1987)).

Other demyelinating diseases have shown that iron homeostasis is significantly disrupted at the cellular level, including demyelination associated with HIV infection (siderotic microglia in the demyelinated regions; Gelman, B., et al., Arch. Pathol. Lab. Med., 116, 509 (1992)), Pelizaeous-Merzbacher (reduced transferrin levels and appearance of biochemically abnormal transferrin; Koeppen, A., et al., J. Neurol. Sci., 84, 315 (1988), and Jaeken, J., et al., Clin. Chim. Acta, 144, 245 (1984)), and progressive rubella panencephalitis (iron deposits in cells in centrum ovale; Valk, J., Magnetic Resonance of Myelin, Myelination, and Myelin Disorders. Springer-Verlag, New York, (1989)).

Alzheimer's Disease (AD)

Amyloid proteins (commonly known in the art, and as is intended in the present specification, is a form of aggregated protein that share several properties) are the main precursors and components extracted from the neuritic and vascular amyloid of AD brains. The presence of large numbers of beta-amyloid-containing plaques and tangles is used as a definitive diagnosis of AD (Bondareff, W., Psychiatr. Annals, 14,179 (1984)). Senile plaques and neurofibrillary tangles present in an individual correlates well with the extent of dementia in AD patients (Wilcock, G. K. Esiri, M. M., J. Neurol. Sci, 56, 343 (1982)). These proteins are usually most concentrated in regions of high neuronal cell death, and may be present in various morphologies, including amorphous deposits, neurophil plaque amyloid, and amyloid congophilic angiopathy (Masters, C. L., et al., EMBO J., 4:2757 (1985); Masters, C. L., et al., Proc. Natl. Acad. Sci. USA 82: 4245 (1985)). Thus, this brain amyloidosis (any disease characterized by the extracellular accumulation of amyloid in various organs and tissues of the body) strongly implicates this protein in the etiopathogenesis of AD (Mann. D. M. A., Hardy, J., Neurobiol. Aging, 7, 444 (1986); and Sniewski, H. M., et al., Alzheimer's Disease, A Cerebral Form of Amyloidosis. In: Immunology and Alzheimer's disease, Pouplard-Bartelaix. A., Emile, J., and Christen, Y., eds., Springer-Verlag, Paris (1988)). Plaques and tangles accumulate in highest numbers in the hippocampus and the neocortex (Roth, M., Br. Med. Bull., 42, 42 (1986)). Functionally, these regions play important roles in memory and cognitive function. These functions are exceptionally degraded in Alzheimer patients and symptomatically represent the hallmark in the earlier stages of this disease. Hence, the presence of beta-amyloid proteins in Alzheimer brains would disrupt neuronal function. The extracellular deposits of amyloid create physical barriers between neuritis, which significantly hinder and impede normal synaptic structure and normal physiological functions.

It was found that beta-amyloid produce oxidative stress through the generation of copious amounts of reactive oxygen species (ROS), which include hydroxyl radicals and hydrogen peroxide (Multhaup, G., et al., Science, 271, 1406 (1996)). The production of these ROS follows a Fenton-phenotypic pathway. The hydroxyl radical formed is very reactive and rapidly attacks the beta-amyloid, causing it to polymerize, and further accentuate the neuronal damage. Thus, an effective treatment for AD is the present invention, which upon administering an effective amount of inhibitors to said subject would interfere and/or reduce metal-mediated production of ROS.

Several studies have implicated iron imbalance in AD. Laboratory studies on postmortem AD brains have shown that iron was a significant component of senile plaque, and iron encrustation of blood vessels in AD brains is a common observation. Particularly, in the CA1 region of the hippocampus, amygdala, nucleus basalis of Meynert and the cerebral cortex (Lovel, M. A., et al., J. Neurol. Sci, 158, 470 (1998); Beard, J. L., et al., Nutr. Rev., 51, 157 (1993); Fisher, P., et al., Life Sci., 60, 2273 (1997); Shoham, S., et al., Cell Mol. Biol., 46, 743 (2000); and Hautot, D., et al., Proc. R. Soc. Lond. B, 270, S62 (2003)).

The proceeding example teaches the methods of the present invention and the use of the disclosed inhibitors. This example is only intended for illustrative purposes and is in no way limit the scope of the disclosed invention. Artisans of ordinary skill would be able to use the methods described by the following example to practice the full scope of the present invention and realize its merits.

### EXAMPLE

The aerobic acidophilic iron-oxidizing bacterium T. ferrooxidans was isolated from a coal mine drainage, and its recovery was reported by Colmer and Hinkle in 1947 (Colmer, A. R., & Hinkle, M. E., Sci., 106, 253 (1947)). Following its discovery, this microorganism was soon associated with the commercial extraction of copper and uranium from ores by microbial leaching through oxidation and acidification. T. ferrooxidans is a motile, single-pole flagellated, aerobic, chemolithotrophic, chemoautotrophic (CO₂ fixers and possess Calvin cycle enzymes), rod-shaped cell (0.3-0.4 μm across by 0.5-1.0 μm long), gram-negative, and its division time is variable with conditions (Brierley, L. C., CRC Crit. Rev. Microbiol., 28, 207 (1978); Ingeldew, W. J., Biochim. Biophys. Acta, 683, 89 (1980); and Murr, L. E., Miner. Sci. Eng., 12, 121 (1980)). T. ferrooxidans is a chemoautotroph that gains energy by oxidative phosphorylation, nitrogen from N₂ in the air, and carbon exclusively from CO₂ fixation via ribulose-1,5-bisphosphate carboxylase and a classic Calvin cycle (Rawlings, D. E., ed., Biomining: Theory, Microbes and Industrial Processes (Springer, Berlin), 1997; Holmes, D. S., in Bioconversion of Waste Materials to Industrial Products, ed. Martin, A. M. (Blackie Academic and Professional, London), 2nd ed., 517 (1998); and Rawlings, D. E. & Kusano, T., Microbiol. Rev. 58, 39 (1994)). It derives energy from oxidation of reduced sulfur to H₂SO₄ and oxidation of Fe⁺² to Fe⁺³ which precipitates as insoluble Fe(OH)₃. T. ferrooxidans strains have single chromosomes, which may range in genome size from 2.2 Mbp to 2.9 Mbp (Amils, R., et al., Biochimie, 80, 911 (1998)).

Other characteristics of T. ferrooxidans are given in Table 1, below. It belongs to the thiobacilli group, and is a major microorganism important in ore leaching operations, where it solubilizes, for example, sulfide minerals and produces copious amounts of sulfuric acid, releasing metal ions into leach solutions (Batarseh, K. I., et al., AIChEJ, 40, 1741 (1994); and Batarseh, K. I., et al., Chem. Eng. Com., 155, 229 (1997)).

This ability is achieved by the oxidation of ferrous iron to ferric iron as the energy-generating reaction for growth. T. ferrooxidans uses the natural proton gradient in its low pH environment to generate ATP via ATP synthase, then eliminates excess cytoplasmic protons by coupling oxygen reduction to ferrous iron oxidation using rusticyanin, cytochrome c and cytochrome al (all in the cytoplasmic membrane). T. ferrooxidans is generally assumed to be obligately aerobic, but under anaerobic conditions, T. ferrooxidans can be grown on elemental sulfur using ferric iron as an electron acceptor. These results indicate that T. ferrooxidans can be considered a facultative anaerobe playing an important role in the iron and sulfur cycles in acidic environments.

TABLE 1
Characteristics of T. ferrooxidans*.

| Condition | Characteristic |
|---|---|
| Optimum growth pH | 1.3-4.5 |
| Temperature range | 10-37° C. |
| Optimum temperature | 30-35° C. |
| Motility | 0 to several polar or peritrichous flagella |
| Gram staining | Gram-negative |
| Spore formation | None |
| Trophy | Obligate chemolithoautotroph |
| Energy pathway | Oxidation of Fe²⁺ and reduced sulfur |
| Oxygen requirements | Obligate aerobe |
| Electron acceptor | Oxygen |
| Nitrogen source | Ammonium salts, nitrate, fix dinitrogen |
| Oxygen requirements | Obligate aerobe |

*Stanley, J. T., et al., Bergeys Manual of Systematic Bacteriology, V. 3, Williams & Wilkins, Baltimore, MD (1989).

The energy-generating system which resides in the cell envelope (Ingeldew, W. J., et al., Proc. Soc. Gen. Microbiol., 4, 74 (1977)) is mediated through an electron-transport chain between two half reactions which are catalyzed by T. ferrooxidans. Physiologically, the following events occur: Fe+2 is oxidized according to: 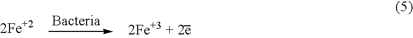
consequently, O₂ can be reduced to H₂O by: 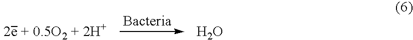
where the electrons transferred produce enough energy to insure the formation of ATP when coupled to oxidative phosphorylation. The reductant contains no H and linkage of Fe couple and O₂/H₂O couple consumes protons. This proton consumption occurs on the cytoplasmic side of bacterial membrane whilst Fe oxidation occurs in periplasm which contains a specific first redox intermediate rusticyanin. Large turnover of ferrous iron oxidation is needed to obtain very little energy. This has resulted in the evolution of a diversity of initial redox components and enzymes specific to the electron donor and it is very different from the NADH oxidase in mitochondria.

Rusticyanin is a small type I blue copper-containing metalloprotein, composed of a core β-sandwich fold, which has 1 atom of copper per molecule of protein, and a molecular mass of 16,551 Da (Ronk, M., et al., Biochemistry, 30, 9435 (1991)). It is found in abundance in the periplasmic space, and can constitute as much as 5% of the total soluble proteins synthesized by cells of T. ferrooxidans that have been grown autotrophically on ferrous iron (Cox, J. C. & Boxer, D. H., Biochem. J, 174, 497 (1978)). This protein displays unusual acid stability (to pH<2.0) and the highest redox potential (680 mV) of any member of the cupredoxin family (Walter, R. L., et al., J. Mol. Biol., 263, 730 (1996)). In rusticyanin, the protein folds as a beta-sandwich, consisting of a six-stranded and a seven-stranded beta-sheet, typical for a cupredoxin (Harvey, I., et al., Acta Crystallographica, D54, (4), 62 (1998)). Unusually, rusticyanin possesses an N-terminal extension consisting of 38 amino acid residues which contains an alpha-helix. This has been proposed to be involved in the acid stability of this protein. The copper ion is coordinated by a cluster of four conserved residues (His 85, Cys138, His143, Met148) arranged in a distorted tetrahedral geometry.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

1. A method of treating, reducing the risk of, or slowing the onset of a neurological disorder or pathological condition, the method comprising the step of administering to a subject in need thereof an inhibitor of formation of free radical or reactive oxygen species in the subject.

2. The method of claim 1, wherein the inhibitor reduces a risk of cellular damage mediated by free radical or reactive oxygen species.

3. The method of claim 1, wherein the inhibitor inhibits the formation of .O⁻₂, H₂O₂ or .OH.

4. The method of claim 1, wherein the inhibitor inhibits the formation of hydroxyl radicals.

5. A method of treating, reducing the risk of, or slowing the onset of a neurological disorder or pathological condition, the method comprising the step of administering to a subject in need thereof an inhibitor of formation of free radical or reactive oxygen species in the subject, wherein the inhibitor is a bacterial metalloprotein.

6. The method of claim 5, wherein the bacterial metalloprotein is found in chemolithotrophic bacteria.

7. The method of claim 5, wherein the bacterial metalloprotein is found in Thiobacillus ferrooxidans.

8. The method of claim 5, wherein the metalloprotein is a type I blue copper protein.

9. The method of claim 5, wherein the metalloprotein is azurin, plastocyanin, amicyanin, stellasyanin, umecyanin, or rusticyanin, or mixtures thereof.

10. The method of claim 5, wherein the metalloprotein is rusticyanin.

11. The method of claim 5, wherein the metalloprotein promotes conversion of ferrous iron to ferric iron in the subject.

12. The method of claim 5, wherein the metalloprotein is administered in an effective amount to have an inhibitory effect on the formation of .O⁻₂, H₂O₂ or .OH.

13. The method of claim 5, further comprising the step of administering a scavenger of reactive oxygen species.

14. The method of claim 5, wherein the scavenger of reactive oxygen species is catalase, superoxide dismutase (SOD), ascorbate peroxidase, or glutathione peroxidase.

15. The method of claim 1 wherein the neurological disease or pathological condition is aging pathology, septic shock, ischemia, diabetes, arthritis, asthma, Alzheimer's disease, Parkinson's disease, multiple sclerosis, cirrhosis, allograft rejection, encephalomyelitis, meningitis, pancreatitis, peritonitis, vasculitis, lymphocytic choriomeningitis, glomerulonephritis, an ophthalmologic disease, chronic fatigue syndrome, acute renal failure, stroke, cancer, cachexia, myocarditis, an autoimmune disorder, eczema, psoriasis, urticaria, cerebral ischemia, systemic lupus erythematosis, AIDS, AIDS dementia, chronic neurodegenerative disease, cystic fibrosis, amyotrophic lateral sclerosis, Huntington's disease, hyperphagia, neuroblastoma, hematologic cancer, liver disease, drug-induced lung injury, transplant rejection, vascular aneurysm, myocardial infarction, or cerebral vasospasms.

16. A composition comprising an inhibitor of formation of free radical or reactive oxygen species and a pharmaceutically acceptable carrier.

17. The composition of claim 16, wherein said inhibitor is a bacterial metalloprotein.

18. The composition of claim 17, wherein the bacterial metalloprotein is a metalloprotein that is found in chemolithotrophic bacteria.

19. The composition of claim 17, wherein the bacterial metalloprotein is a metalloprotein that is found in Thiobacillus ferrooxidans.

20. The composition of claim 17, wherein the bacterial metalloprotein is a type I blue copper protein.

21. The composition of claim 17, wherein the bacterial metalloprotein is rusticyanin.

22. The composition of claim 17, wherein the bacterial metalloprotein is azurin, plastocyanin, amicyanin, stellasyanin, umecyanin, or rusticyanin, or mixtures thereof.

23. The composition of claim 17, wherein the bacterial metalloprotein promotes conversion of ferrous iron to ferric iron.

24. The composition of claim 17, wherein the bacterial metalloprotein inhibits the formation of a reactive oxygen species.

25. The composition of claim 17, wherein the bacterial metalloprotein inhibits the formation of .O⁻₂, H₂O₂ or .OH.

26. The composition of claim 17, wherein the pharmaceutically acceptable carrier comprises a biological buffer

27. The composition of claim 17, wherein the pharmaceutically acceptable carrier comprises a biological buffer that lacks available aqua coordination sites.

28. The composition of claim 17, wherein the pharmaceutically acceptable carrier comprises a diethylenetriamine pentaacetic acid.

29. The composition of claim 17, further comprising DNA-intercalating agents.

30. The composition of claim 17, further comprising agents that block activity of topoisomerase I and topoisomerase II.

31. The composition of claim 17, further comprising cytokines, antimicrobial agents, hormones, sedatives, hypnotics, tranquilizers, topically active drugs, vasodilating substances, biological substances that affect tissue formation and tissue stabilization, sunscreens, cleansing agents or antiperspirants.

32. The composition of claim 17, further including at least one additional electron source.

33. The composition of claim 17, wherein the additional electron source is a thiol, ascorbate, paraquat, anthraquinone, quinone, semiquinone, redox-activating drugs, antibiotic agent, antitumor agent, bleomycin, amsacrine, mitomycin C, adriamycin, actinomycin D, daunomycine, neocarsinostatin, steptonigrin, elliptinium acetate, or mixtures thereof.

34. The composition of claim 17, further comprising a scavenger of reactive oxygen species.

35. The composition of claim 17, further comprising the scavenger of reactive oxygen species is catalase, superoxide dismutase (SOD), ascorbate peroxidase, or glutathione peroxidase.

36. The composition of claim 17, further comprising at least one metal chelator.

37. The composition of claim 17, further comprising an antiviral agent.
